(19) United States
(12) Patent Application Publication
(10) Pub. No.: US 2025/0213437 A1
(43) Pub. Date: Jul. 3, 2025

(54) STABLE SUSPENSION OF MICROFIBROUS CELLULOSE

(71) Applicant: MINASOLVE SAS, Beuvry-La-Forêt (FR)

(72) Inventors: Markus NAHRWOLD, Minden (DE); Victoria RENOULT, Louvain-La-Neuve (BE)

(21) Appl. No.: 18/850,751

(22) PCT Filed: Mar. 31, 2023
(86) PCT No.: PCT/EP2023/058520

§ 371 Date: Sep. 25, 2024

(30) Foreign Application Priority Data
Apr. 1, 2022 (EP) 22166319.8

(51) Int. Cl. A61K 8/04; A61K 8/34; A61K 8/73; A61Q 1/02; A61Q 5/02

(52) U.S. Cl. CPC A61K 8/044; A61K 8/345; A61K 8/731; A61Q 1/02; A61Q 5/02

(57) ABSTRACT

The current invention relates to a stable suspension comprising: microfibrous cellulose, microfibrous hemicellulose or mixtures thereof (MF(H)C), in an amount of 1 to 10 percent by weight of the suspension; at least one natural thickening agent, in an amount up to 2 percent by weight of the suspension; at least one polyol, in an amount up to 20 percent by weight of the suspension; and water, in an amount of 50 to 98 percent by weight of the suspension. In a second aspect, the present invention relates to a method for producing aforementioned stable suspension. In a third aspect, the present invention relates to a use of aforementioned stable suspension in a cosmetic, pharmaceutical, dermatological or hygienic preparation. In a fourth and fifth aspect, the present invention relates to a cosmetic, pharmaceutical, dermatological or hygienic product comprising aforementioned stable suspension and a method for the production thereof.

### FIELD OF THE INVENTION

The present invention relates to a stable suspension of microfibrous cellulose and/or microfibrous hemicellulose, methods for production thereof and their use in cosmetic, pharmaceutical, dermatological or hygienic preparations.

### BACKGROUND

There is a continuous need for sustainable functional ingredients that can stabilize water-based formulations or improve their physical and sensory properties. Of particular interest are abundantly available natural polymers, such as cellulose or hemicellulose. In many cases the structure and functionality of the natural materials can be modified in order to obtain products with superior performance.

Natural cellulose and hemicellulose fibers consist of polysaccharide chains. Several chains are stacked and form linear fibrils which are held together by hydrogen bonds. Mechanical shear, commonly combined with chemical, enzymatic or fermentative pre-treatment, can break down the natural linear fibers into a three-dimensional network of microfibrils. Such microfibrous or “microfibrillated” cellulose (MFC) is characterized by having a large surface area per volume, on the order of 1000 times larger than that of ordinary pulp fibers.

The special structure of MFC leads to a high water-binding capacity and the ability to form gel-like aqueous suspensions. These properties are of interest for various technical applications. MFC is suitable, for example, as a binder, thickener, rheology modifier, wetting agent and co-emulsifier. Its film-forming activity and stabilizing effect on multiphase liquid formulations makes MFC particularly interesting for personal care applications. Examples of cosmetic applications include skin or hair conditioning, reduction of oiliness, as well as stabilization of foam, emulsions, or suspensions.

MFC is chemically resistant and therefore tolerant towards a wide range of pH values and temperatures as well as towards high concentrations of salt and solvent. It is therefore a particularly versatile ingredient for a wide range of products in various industries.

Despite its interesting properties, the application of MFC on an industrial scale brings several disadvantages:
Aqueous suspensions of MFC are only stable to a limited extent. The typical network structure may get lost at least partially during prolonged storage. Such reaggregation of the fibers causes MFC to lose its beneficial properties. Similar phenomena can be observed following a drying step where the amount of water in the MFC is reduced. Therefore, MFC usually needs to be freshly activated before or during use by subjecting it to high shear forces. This requires an additional mixing step with systems such as rotor-stator mixers. Therefore, MFC is hardly suitable for processes that do not involve or allow high shear mixing. An additional high-speed mixing step also increases production costs since high shear mixers are usually more expensive than low shear mixers as well as harder to clean and to maintain.
The application of high shear forces on industrial scale means that any mixing process must be adapted to the existing mixing equipment. Careful optimization of critical parameters such as mixing speed and time may be required. Switching from one type of equipment to another may require repeated process optimization. Upscaling from laboratory scale to industrial scale can be problematic and time consuming. All these additional measures extend the time to market and increase the risk of quality deviations.
During prolonged storage, aqueous MFC suspensions tend to sediment, i.e., the fibers gradually settle, and a water layer forms on the surface. This phenomenon is also known as syneresis. It leads to an inconsistent composition, which is problematic for use in industrial processes.
Freezing and subsequent thawing of aqueous MFC suspensions leads to a breakdown of their three-dimensional fiber network (see also WO2018/002446). This can be disadvantageous, since the MFC suspensions lose their properties when being stored or transported in cold climates.
The high water-content of MFC suspensions can also lead to a short shelf-life because such compositions usually favor the growth of micro-organisms. The addition of preservatives or the use of disinfection procedures may be required, resulting in additional costs and regulatory hurdles.

WO2018/002445 teaches that the high moisture content of MFC gels leads to a short shelf-life. The authors also point out that a removal of water from microfibrillated cellulose causes aggregation of fibrils and hornification, yielding a dramatic loss of the intended properties upon re-suspending the product in water. Solid dried MFC therefore needs to be reactivated by application of high shear force.

WO2018/002445 further discloses a composition comprising fibrillated parenchymal cellulose, at least one liquid activator, and optionally water. The activator optionally comprises glycerol, sorbitol, polyol, polyol mixture, polyol solution with up to 40% water, or polyethylene glycol with a molecular weight below 1000 g/mol. However, WO2018/002445 does not teach that the microfibrillated cellulose can be made storage stable in a gel form with high water content. It also does not disclose a stable gel that withstands heat stress or cold stress and can be used as is without the need for activation through application of high shear force.

EP3081208 A1, WO2010003860 and WO0016889A2 describe the use of MFC in liquid cosmetic and cleaning applications. Compositions containing MFC described therein are typically subjected to at least one mixing step using a rotor-stator mixer, which exerts a high shear force on the formulation.

The present invention aims to resolve at least some of the problems and disadvantages mentioned above by providing a stable suspension of microfibrous cellulose and/or microfibrous hemicellulose.

### SUMMARY OF THE INVENTION

The present invention and embodiments thereof serve to provide a solution to one or more of above-mentioned disadvantages. To this end, the present invention relates, in a first aspect, to a stable suspension of microfibrous cellulose, microfibrous hemicellulose or mixtures thereof (MF(H)C) according to claim 1.

This stable suspension has the following advantages: a uniform semi-solid to semi-liquid paste or gel is obtained, the suspension is stable in warm or cold conditions with no separation during storage, a network of MFC fibers is formed upon suspension in water or aqueous mixtures with low shear, the suspension is resistant to microbial infestation, and the suspension gives the possibility to be used as an ingredient in aqueous formulations without the need of high shear mixing. Furthermore, the performance and a uniform composition of aqueous MF(H)C suspensions during storage at high or low temperatures is maintained, and long shelf life of aqueous MF(H)C suspensions due to self-preserving properties against microbial attack is ensured.

Preferred embodiments of the stable suspension are shown in any of the claims 2 to 10.

In specific preferred embodiments, the present invention comprises natural polyol(s) and natural microfibrous cellulose and/or microfibrous hemicellulose for achieving the advantages mentioned above by using only sustainable and bio-based components.

In a second aspect, the present invention also relates to a method according to claim 11. The inventors have surprisingly found that dispersing the natural thickening agent into the polyol prior to mixing this dispersion with the aqueous suspension comprising MF(H)C improves mixing and reduces difficulties obtaining a homogeneous mixture of all components.

In a third aspect, the present invention also relates to a use of a stable suspension according to claim 12.

This use in cosmetic, pharmaceutical, dermatological or hygienic preparations results in cosmetic, pharmaceutical, dermatological or hygienic products which have the following advantages: stabilization of heterogeneous mixtures by supporting the suspension of particles, oil droplets or gas bubbles in aqueous media, uniform distribution of pigment particles on surfaces, resulting in e.g., intensification of coloration, skin and/or hair conditioning effects, shear-thinning effect, resulting in a rich texture, provision of a smooth, non-oily skin feel.

In a fourth aspect, the present invention also relates to a cosmetic, pharmaceutical, dermatological, or hygienic product according to claim 13.

This cosmetic, pharmaceutical, dermatological or hygienic product has at least one of the following advantages: a uniform opaque gel without high shear mixing can be obtained, a whiter emulsion with less oily skin feel and a richer texture can be obtained, and more intense color can be achieved on the skin by pigment suspensions.

A preferred embodiment of the product is shown in claim 14.

In a fifth aspect, the present invention also relates to a method according to claim 15. More particular, the method as described herein provides that a high-speed mixing step which increases production costs since high shear mixers are usually more expensive than low shear mixers as well as harder to clean and to maintain, is redundant. In addition, the present invention enables the use of MF(H)C in products where mixing under high shear is not possible due to the incompatibility of certain ingredients with high shear forces.

### DETAILED DESCRIPTION OF THE INVENTION

The present invention concerns a stable suspension of microfibrous cellulose and/or microfibrous hemicellulose.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” or “include”, “including”, and “includes” as used herein are inclusive or open-ended terms that specify the presence of what follows e.g. component and do not exclude or preclude the presence of additional, non-recited components, features, element, members, steps, known in the art or disclosed therein.

“Contain”, “containing”, and “contains” or “consists”, “consisting of” and “consisted of” are exclusive or close-ended terms that specify the presence of what follows e.g. component and excludes or precludes the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

“Microfibrous cellulose, microfibrous hemicellulose or mixtures thereof” is synonymous with “MF(H)C” and refers to at least one chosen from the list of: microfibrous cellulose (MFC), microfibrous hemicellulose (MFHC) or a mixture thereof, i.e., both MFC and MFHC.

The term “suspension”, as used in the text, refers to a mixture in which the suspended (MF(H)C) does not dissolve, but gets suspended throughout the bulk of the solvent (water) and floats around freely in the medium. The internal phase (solid, MF(H)C) is dispersed throughout the external phase (fluid, water), preferably aided by mechanical agitation.

The term “homogeneous”, as used in the text, is defined to mean that the suspension is uniform in concentration and visual appearance throughout the entire suspension.

The term “stable”, as used in the text, refers to a uniform semi-solid to semi-liquid suspension which is stable in warm or cold conditions with no separation during storage, such as syneresis or settling of the solids (MF(H)C).

The term “syneresis”, as used in the text, refers to the expulsion of a liquid from a gel, resulting in a heterogeneous mixture.

“C5-diol”, “1,2-pentanediol”, and “pentylene glycol” are synonymous and refer to the chemical compound with CAS-number 5343-92-0.

“C8-diol”, “1,2-octanediol”, and “caprylyl glycol” are synonymous and refer to the chemical compound with CAS-number 1117-86-8.

In a first aspect, the invention relates to a stable suspension, comprising microfibrous cellulose, microfibrous hemicellulose or mixtures thereof (MF(H)C).

In a particularly preferred embodiment of the invention, said stable suspension comprises:
A. microfibrous cellulose, microfibrous hemicellulose or mixtures thereof (MF(H)C), in an amount of 1 to 10 percent by weight of the suspension;
B. at least one natural thickening agent, in an amount up to 2 percent by weight of the suspension;
C. at least one polyol, in an amount up to 20 percent by weight of the suspension; and
D. water, in an amount of 50 to 98 percent by weight of the suspension.

This stable suspension has the following advantages: a uniform semi-solid to semi-liquid paste or gel is obtained, the suspension is stable in warm or cold conditions with no separation during storage, a network of MFC fibers is formed upon suspension in water or aqueous mixtures with low shear, the suspension is resistant to microbial infestation, and the suspension gives the possibility to be used as an ingredient in aqueous formulations without the need of high shear mixing. Furthermore, the performance and a uniform composition of aqueous MF(H)C suspensions during storage at high or low temperatures is maintained, and long shelf life of aqueous MF(H)C suspensions due to self-preserving properties against microbial attack is ensured.

In a preferred embodiment of the invention, the stable suspension comprises microfibrous cellulose (MFC).

In a preferred embodiment of the invention, the stable suspension comprises MF(H)C, in an amount of 1 to 5 percent by weight of the suspension, preferably in an amount of 2 to 4 percent by weight of the suspension, more preferably in an amount of 2 to 3 percent by weight of the suspension, even more preferably between 2.4 and 2.9 percent by weight of the suspension.

This concentration of the microfibrous cellulose and/or microfibrous hemicellulose in the suspension is preferred in order to facilitate an efficient mixing and the incorporation of further solid and/or liquid components.

The MF(H)C used as a raw material for the stable suspensions according to the invention can be microfibrillated from natural fibers by various technologies, including but not limited to one of the following or any combination of the following technical steps:
chemical and/or physical separation of cellulose from other components, for example, the removal of lignin via the kraft pulp/sulfate process under alkaline conditions or the sulfite process under acidic conditions for wood to cellulose conversion; and/or
bleaching of the crude cellulose and/or hemicellulose fibers, preferably using chlorine-free technologies; and/or
partial enzymatic hydrolysis of the cellulose and/or hemicellulose by cellulase treatment; and/or
physical application of high shear forces to the cellulose/hemicellulose pulp.

In a preferred embodiment of the invention, said MF(H)C is microfibrillated from natural fibers by a process comprising only the physical application of high-shear forces and does not involve any chemical, biotechnological, or enzymatic conversions of the natural fibers.

In this way, there is no need for additional use of enzymes, chemicals or other products or solvents and the aforementioned advantages of the suspension can be achieved by using only sustainable components.

In another preferred embodiment of the invention, said MF(H)C is obtained by a process comprising the steps of:
i. subjecting natural fibers, for example vegetable pulp, to chemical and/or enzymatic treatment resulting in partial degradation and/or extraction of pectin and hemicellulose; and
ii. subjecting the material resulting from step i. to a high shear process to yield a particulate material having a volume-weighted median dimension within the range of 25-75 μm, as measured by laser diffractometry.

The term “Laser diffractometry”, as used in the text, is also known as “laser diffraction analysis”, and refers to a technology that utilizes diffraction patterns of a laser beam passed through any object ranging from nanometers to millimeters in size to quickly measure geometrical dimensions of a particle. This process does not depend on volumetric flow rate, which is the number of particles that passes through a surface over time.

Cellulose fibrils are known to aggregate less and form a less compact structure in the presence of hemicellulose (Wan et al., Bioresource Technology 2010, vol 101/issue 12, 4577-4583). In a preferred embodiment, the MF(H)C therefore comprises at least 10% hemicellulose, more preferred at least 20% hemicellulose, most preferred more than 20% hemicellulose.

An even more preferred process for the production of microscale fiber material, comprises the following process steps:
mechanically pre-shredding pulp substantially without adding a liquid, preferably without adding a liquid so that the mean fiber length of the pulp is in the range of 0.025 mm-6 mm;
dispersing the dry, mechanically pre-shredded pulp in a liquid;
finely comminuting the pulp dispersed in the liquid such that a fine comminution mixture is formed, which contains the liquid and the fiber material with micro-scale fibrous agglomerates.

The average length of the microfibrous cellulose and/or microfibrous hemicellulose is preferably in the range of 500 nm to 1000 μm, more preferably in the range of 500 nm to 600 μm, and more preferably in the range of 500 nm to 200 μm.

In a preferred embodiment of the invention, the natural fibers are derived from plant materials or microorganisms, preferably from plant materials, more preferably from plant pulp, even more preferably from hardwood and/or softwood-based pulp. In another embodiment of the invention, the natural fibers are derived from waste plant materials, such as by-products of food processing or agriculture. Non-limitative examples of suitable waste materials are sugar beet pulp, sugarcane bagasse, cassava pulp, potato pulp, citrus peel, as well as stems and/or leaves of other plants. In a further preferred embodiment of the invention, the natural fibers of any of the previous embodiments comprise cellulose and/or hemicellulose, preferably cellulose and hemicellulose.

In a preferred embodiment of the invention, the stable suspension comprises at least one natural thickening agent, in an amount up to 2 percent by weight of the suspension, preferably in an amount up to 1 percent by weight of the suspension, preferably in an amount between 0.1 and 1 percent by weight of the suspension, preferably in an amount between 0.2 and 1 percent by weight of the suspension, more preferably, in an amount between 0.25 and 1 percent by weight of the suspension, even more preferably in an amount between 0.3 and 1 percent by weight of the suspension, even more preferably in an amount between 0.25 and 0.5 percent by weight of the suspension, most preferably in an amount of about 0.4 percent by weight of the suspension.

The inventors have found that lower concentrations of the natural thickening agent result in syneresis. A minimum of 0.1 wt. %, preferably 0.2 wt. %, more preferably 0.3 wt. % natural thickening agent is advantageous to obtain a stable gel. A maximum of 2 wt. % xanthan gum, preferably a maximum of 1 wt. % is preferred, because higher concentrations increase the viscosity, so the gel is harder to handle, and it takes longer to disperse it in water.

The at least one natural thickening agent is preferably a poly-saccharide-based thickener, such as natural gums or cellulose derivatives. Examples for natural gums comprise, Agar, Alginic acid and its salts, beta-glucan, Carrageenan, Cassia gum, Dammar gum, Fenugreek gum, Gellan gum, gelatin, Glucomannan, Guar gum, Gum Arabic (Acacia Senegal gum), Gum ghatti, Karaya gum (Gum tragacanth), Konjac mannan gum, Locust bean gum, Psyllium seed husk, Caesalpinia spinosa (Tara) gum, Xanthan gum. Examples for cellulose-based thickeners comprise carboxymethyl cellulose (cellulose gum), methylcellulose, ethyl cellulose, hydroxyethyl cellulose, hydroxypropyl cellulose, methyl ethyl cellulose, hydroxyethyl methylcellulose, hydroxypropyl ethyl cellulose, ethyl hydroxyethyl cellulose, carboxymethyl hydroxyethyl cellulose, methyl hydroxypropyl cellulose.

In a preferred embodiment of the invention, the natural thickening agents are chosen from the list of: Xanthan gum, Locust bean gum, Gellan gum, Guar gum or any combinations thereof. In a more preferred embodiment of the invention, the stable suspension comprises one natural thickening agent, preferably chosen from the list of: Xanthan gum, Locust bean gum, Gellan gum, Guar gum, more preferably Xanthan gum.

In a preferred embodiment of the invention, the stable suspension comprises at least one polyol, in an amount between 0.5 and 20 percent by weight of the suspension, preferably in an amount between 1 and 20 percent by weight of the suspension, more preferably in an amount between 2 and 20 percent by weight of the suspension, more preferably in an amount between 2.5 and 20 percent by weight of the suspension, more preferably in an amount between 3 and 20 percent by weight of the suspension, more preferably in an amount between 3 and 10 or between 19 and 20 percent by weight of the suspension, even more preferably in an amount of about 3, 5 or 10 percent by weight of the suspension or between 19 and 20 percent by weight of the suspension.

In a more preferred embodiment of the invention, the stable suspension comprises at least one polyol, in an amount between 3 and 10 percent by weight of the suspension.

The inventors have found that the stable suspensions comprising at least one polyol in this weight range, are physically stable after a freeze-thaw test (−20° C. to +20° C.).

In a preferred embodiment of the invention, the stable suspension comprises at least one polyol, in an amount of at most 20 percent by weight of the suspension, preferably in an amount of at most 17.5 percent by weight of the suspension, preferably in an amount of at most 15 percent by weight of the suspension, preferably in an amount of at most 12.5 percent by weight of the suspension, more preferably in an amount of at most 10 percent by weight of the suspension. Lower quantities are advantageous due to the cost of polyols.

The at least one polyol is preferably selected from the list of diols or triols comprising a branched or non-branched chain of 3 to 12 carbon atoms or any mixture thereof. In a preferred embodiment of the invention the at least one polyol is selected from the list of: glycerol, 1,2-propanediol, 1,3-propanediol, butylene glycol, 1,2-pentanediol, isopentyldiol, 1,2-hexanediol, hexylene glycol, 1,2-heptanediol, 1,2-octanediol, 1,2-nonanediol, 1,2-decanediol, 1,2-undecanediol, methylheptylglycerin, ethylhexylglycerin and 1,2-dodecanediol or a mixture thereof, preferably the at least one polyol is selected from the list of: glycerol, 1,2-pentanediol, 1,2-octanediol or any mixtures thereof.

In a preferred embodiment of the invention, the stable suspension comprises one or two polyols. In a further embodiment the one or two polyols are chosen from the list of glycerol, 1,2-propanediol, 1,3-propanediol, butylene glycol, 1,2-pentanediol, isopentyldiol, 1,2-hexanediol, hexylene glycol, 1,2-heptanediol, 1,2-octanediol, 1,2-nonanediol, 1,2-decanediol, 1,2-undecanediol, methylheptylglycerin, ethylhexylglycerin and 1,2-dodecanediol or a mixture thereof, preferably the one or two polyols are selected from the list of: glycerol, 1,2-pentanediol, 1,2-octanediol. In an even more preferred embodiment, the stable suspension comprises a polyol chosen from the list of: 1,2-pentanediol or a mixture of glycerol and 1,2-octanediol.

When the stable suspension comprises a mixture of glycerol and 1,2-octanediol, the ratio by weight of glycerol to 1,2-octanediol is preferably between 40/1 and 5/1. In one further embodiment the ratio by weight of glycerol to 1,2-octanediol is between 40/1 and 35/1, preferably between 40/1 and 36/1, more preferably between 40/1 and 37/1, even more preferably between 39/1 and 37/1. In another further embodiment the ratio by weight of glycerol to 1,2-octanediol is between 25/1 and 5/1, preferably between 20/1 and 5/1, more preferably about 20/1, about 10/1 or about 5/1.

In a further embodiment of the invention, the at least one polyol is produced from renewable feedstock, such as vegetable raw material or carbon dioxide for achieving the advantages mentioned above by using only sustainable and bio-based components.

In an embodiment of the invention the ratio by weight of the at least one polyol and the at least one thickening agent is between 80/1 and 6/1, preferably the ratio by weight of the at least one polyol and the at least one thickening agent is chosen from the list of: about 79/1, about 69/1, about 59/1, about 49/1, about 39/1, about 29/1, about 21/1, about 20/1, about 19/1, about 13.75, about 12.5/1, about 9/1, about 7.5/1 or about 6/1. In a preferred embodiment the ratio by weight of the at least one polyol and the at least one thickening agent is between 14/1 and 10/1.

When the stable suspension comprises a 1,2-pentanediol as polyol, the ratio by weight of the at least one polyol and the at least one thickening agent is between 50/1 and 10/1, preferably about 12.5/1.

In a preferred embodiment the stable suspension comprises the at least one polyol and the at least one natural thickening agent in a combined amount of up to 22 percent by weight of the suspension, preferably in an amount of up to 20 percent by weight of the suspension, more preferably in an amount of up to 10 percent by weight of the suspension, most preferably in an amount up to 6 percent by weight of the suspension.

In a preferred embodiment of the invention the stable suspension has the form of a homogeneous suspension, preferably a homogeneous gel.

In a particularly preferred embodiment of the invention, the stable suspension consists of:
A. microfibrous cellulose, microfibrous hemicellulose or mixtures thereof (MF(H)C), in an amount of 1 to 10 percent by weight of the suspension;
B. at least one natural thickening agent, in an amount up to 2 percent by weight of the suspension;
C. at least one polyol, in an amount up to 20 percent by weight of the suspension; and
D. water, in an amount of 68 to 98 percent by weight of the suspension.

In a particularly preferred embodiment of the invention, the stable suspension consists of:
A. microfibrous cellulose, microfibrous hemicellulose or mixtures thereof (MF(H)C), in an amount of 1 to 10 percent by weight of the suspension;
B. at least one natural thickening agent, in an amount up to 2 percent by weight of the suspension;
C. at least one polyol, in an amount up to 20 percent by weight of the suspension;
D. water, in an amount of 68 to 98 percent by weight of the suspension; and
E. impurities and/or additives in an amount up to 5 percent by weight of the suspension, preferably in an amount up to 3 percent by weight of the suspension, more preferably in an amount up to 2 percent by weight of the suspension, more preferably in an amount up to 1 percent by weight of the suspension, more preferably in an amount up to 0.5 percent by weight of the suspension, more preferably in an amount up to 0.1 percent by weight of the suspension.

In a particularly preferred embodiment of the invention, the stable suspension consists of:
A. microfibrous cellulose, microfibrous hemicellulose or mixtures thereof (MF(H)C), in an amount of 1 to 10 percent by weight of the suspension;
B. at least one natural thickening agent, in an amount of 0.2 to 1 percent by weight of the suspension;
C. at least one polyol, in an amount 3 to 10 percent by weight of the suspension; and
D. water, in an amount of 79 to 95.8 percent by weight of the suspension.

In a particularly preferred embodiment of the invention, the stable suspension consists of:
A. microfibrous cellulose, microfibrous hemicellulose or mixtures thereof (MF(H)C), in an amount of 1 to 10 percent by weight of the suspension;
B. at least one natural thickening agent, in an amount of 0.2 to 1 percent by weight of the suspension;
C. at least one polyol, in an amount 3 to 10 percent by weight of the suspension; and
D. water, in an amount of 79 to 95.8 percent by weight of the suspension; and impurities and/or additives in an amount up to 5 percent by weight of the suspension, preferably in an amount up to 3 percent by weight of the suspension, more preferably in an amount up to 2 percent by weight of the suspension, more preferably in an amount up to 1 percent by weight of the suspension, more preferably in an amount up to 0.5 percent by weight of the suspension, more preferably in an amount up to 0.1 percent by weight of the suspension.

In a particularly preferred embodiment of the invention, the stable suspension consists of:
A. microfibrous cellulose, microfibrous hemicellulose or mixtures thereof (MF(H)C), in an amount of 1 to 10 percent by weight of the suspension;
B. at least one natural thickening agent, in an amount of 0.3 to 1 percent by weight of the suspension;
C. at least one polyol, in an amount 3 to 10 percent by weight of the suspension; and
D. water, in an amount of 79 to 95.7 percent by weight of the suspension.

In a particularly preferred embodiment of the invention, the stable suspension consists of:
A. microfibrous cellulose, microfibrous hemicellulose or mixtures thereof (MF(H)C), in an amount of 1 to 10 percent by weight of the suspension;
B. at least one natural thickening agent, in an amount of 0.3 to 1 percent by weight of the suspension;
C. at least one polyol, in an amount 3 to 10 percent by weight of the suspension; and
D. water, in an amount of 79 to 95.7 percent by weight of the suspension; and impurities and/or additives in an amount up to 5 percent by weight of the suspension, preferably in an amount up to 3 percent by weight of the suspension, more preferably in an amount up to 2 percent by weight of the suspension, more preferably in an amount up to 1 percent by weight of the suspension, more preferably in an amount up to 0.5 percent by weight of the suspension, more preferably in an amount up to 0.1 percent by weight of the suspension.

In a preferred embodiment, said suspension is stable for at least 1 year, more preferably at least 2 years, more preferably at least 3 years, more preferably at least 4 years, more preferably at least 5 years, more preferably at least 8 years, more preferably at least 10 years, more preferably at least 12 years, more preferably at least 15 years, most preferably indefinitely.

In a second aspect the invention relates to a method for producing a stable suspension according to the first aspect of the invention.

In a particularly preferred embodiment of the invention, the method comprises the steps of:
i. providing an aqueous suspension comprising MF(H)C,
ii. dispersing at least one natural thickening agent in at least one polyol, and
iii. adding the dispersion obtained in step ii. to the aqueous suspension comprising MFC under mixing.

In a preferred embodiment of the invention, the method comprises the steps of:
i. providing an aqueous suspension comprising MF(H)C, in an amount of 1 to 10 percent MF(H)C by weight of the stable suspension and 50-98 percent water by weight of the stable suspension,
ii. dispersing at least one natural thickening agent in an amount of up to 2 percent by weight of the stable suspension in at least one polyol in an amount of up to 20 percent by weight of the stable suspension, and
iii. adding the dispersion obtained in step ii. to the aqueous suspension comprising MFC under mixing.

In another preferred embodiment of the invention, the method comprises the steps of:
i. providing an aqueous suspension comprising MF(H)C, in an amount of 3 percent MF(H)C by weight of the aqueous suspension,
ii. dispersing at least one natural thickening agent in an amount of up to 2 percent by weight of the stable suspension in at least one polyol in an amount of up to 20 percent by weight of the stable suspension, and
iii. adding the dispersion obtained in step ii. to the aqueous suspension comprising MFC under mixing.

In even another preferred embodiment of the invention, the method comprises the steps of:
i. providing an aqueous suspension comprising MF(H)C, in an amount of 3 percent MF(H)C by weight of the aqueous suspension,
ii. dispersing at least one natural thickening agent in at least one polyol, in a ratio by weight of the at least one polyol and the at least one thickening agent of between 80/1 and 6/1, and
iii. adding the dispersion obtained in step ii. to the aqueous suspension comprising MFC from step i., preferably in a ratio by weight up to 22:78 of dispersion to aqueous suspension comprising MFC, under mixing.

In a third aspect, the present invention relates to a use of a stable suspension according to the first aspect of the invention in a cosmetic, pharmaceutical, dermatological or hygienic preparation.

This use in cosmetic, pharmaceutical, dermatological or hygienic preparations results in cosmetic, pharmaceutical, dermatological or hygienic products which have at least one of the following advantages: stabilization of heterogeneous mixtures by supporting the suspension of particles, oil droplets or gas bubbles in aqueous media, uniform distribution of pigment particles on surfaces, resulting in e.g., intensification of coloration, skin and/or hair conditioning effects, shear-thinning effect, resulting in a rich texture, provision of a smooth, non-oily skin feel.

In a fourth aspect, the present invention relates to a cosmetic, pharmaceutical, dermatological or hygienic product comprising a stable suspension according to the first aspect of the invention.

This cosmetic, pharmaceutical, dermatological or hygienic product has at least one of the following advantages: a uniform opaque gel without high shear mixing can be obtained, a whiter emulsion with less oily skin feel and a richer texture can be obtained, and more intense color on the skin in pigmented creams can be obtained.

In a preferred embodiment of the invention, said cosmetic, pharmaceutical, dermatological or hygienic product comprises the stable suspension according to the first aspect of the invention in an amount of between 1 and 30 percent by weight of the product, preferably in an amount of between 1 and 20 percent by weight of said product, more preferably between 3 and 20 percent by weight of said product, more preferably in an amount of about 3.3 or an amount of about 18.5.

In an embodiment of the invention, said cosmetic, pharmaceutical, dermatological or hygienic product comprises an additional natural thickening agent, preferably Xanthan gum.

In an embodiment of the invention, said cosmetic, pharmaceutical, dermatological or hygienic product further comprises components chosen from the list of, Coco-Glucoside, Sodium Cocoyl Glutamate, Cocamidopropyl Betaine, Caprylyl Glycol, Glycerin.

Cosmetic, pharmaceutical, dermatological or hygienic products containing stable suspensions according to the invention will also preferably contain water. The formulations may further contain any other functional or active ingredients, such as but not limited to flavours and/or fragrances, fats and/or oils, surfactants, thickeners, emollients, humectants, emulsifiers, chelating agents, gelling agents, binders, texturizing agents, solvents, mineral and/or organic UV filters and/or UVA and/or UVB blocking agents, antioxidants, waxes, polymers, inorganic and/or organic pigments, colouring agents, clays and/or other mineral powders, vegetable materials, natural extracts, essential oils, APIs and other additives commonly used in such formulations. In water-based formulations, it is preferred that about 20 wt % to about 95 wt % water be present therein. The various additives in addition to the water and the preferred antimicrobial systems described herein make up the remaining amount. Preferably, each additive is present in an amount up to about 75% by weight of the total formulation, and more preferably up to about 40% by weight, with the total amount of such additives preferably not exceeding about 50% by weight.

In a preferred embodiment, stable suspensions according to the invention are used in formulations where the continuous phase contains water.

In an embodiment of the invention, said cosmetic, pharmaceutical, dermatological or hygienic product has a pH between 3 and 10, preferably between 4 and 8, more preferably between 5 and 7, most preferably about 5.5.

The cosmetic, pharmaceutical, dermatological or hygienic product can be for example but is not limited to a shampoo, an O/W emulsion or a skin cream, which may comprise pigments.

Further non-limiting examples of cosmetic, pharmaceutical, dermatological or hygienic products include:
a) solutions (aqueous, organic, hydro-alcoholic, hydro-glycolic)
b) suspensions,
c) emulsions (oil in water, water in oil, silicon in water, water in silicon, microemulsions)
d) gels,
e) ointments,
f) pastes,
g) syrups,
h) solids and/or powders,
i) foams,
j) soaps,
k) capsules,
l) perfumes,
m) hydrosols,
n) shampoos,
o) creams,
p) micellar waters,
q) microencapsulated systems,
r) liposome-based systems,
s) wipes and towelettes,
t) any combination of a) to s).

In a fifth aspect, the present invention relates to a method for producing a cosmetic, pharmaceutical, dermatological or hygienic product according to the fourth aspect of the invention.

The method as described herein provides a composition for which that a high-speed mixing or homogenization step is redundant. A high-speed mixing step increases production costs since high shear mixers are usually more expensive than low shear mixers as well as harder to clean and to maintain.

The invention is further described by the following non-limiting examples which further illustrate the invention, and are not intended to, nor should they be interpreted to, limit the scope of the invention.

### EXAMPLES AND FIGURES

The invention is described in more detail in the following examples. The employed microfibrous cellulose was produced by a physical mixing process, particularly a high-shear process.

FIGS. 1-8 show microscopic pictures of several of the suspensions, taken before and after three freeze-thaw cycles of storage at −20° C. for 12 hours, followed by storage at 20° C. for 12 hours.

### COMPARATIVE EXAMPLES 1-3 AND EXAMPLES 4-25

Compositions according to table 1 were prepared according to the following process:
The natural thickening agent was pre-dispersed in the polyol(s) C5-diol and/or C8-diol and/or glycerol.
The dispersion was added to the aqueous MFC Gel under mixing at 10.000 rpm for 3 minutes with a Silverson higher shear rotor/stator laboratory mixer, type L5M-A, equipped with a general-purpose disintegrating head.

The different compositions were subjected to different stress tests:
(1) Centrifugation for 10 min at a spinning rate of 2000 rpm
(2) Three consecutive freeze-thaw cycles of storage at −20° C. for 12 hours, followed by storage at 20° C. for 12 hours.
(3) Storage for 8 weeks in a heated chamber at 50° C.

Following these stress tests, the samples were each inspected for their visual appearance. The results of this examination are summarized in Table 1, where “+” indicates a homogeneous gel, “+/−” indicates slight syneresis, and “−” indicates an inhomogeneous mixture.

TABLE 1

| | Composition of mixtures in % by weight | | | | | | | | | | |
|---|---|---|---|---|---|---|---|---|---|---|---|
| | MFC | | | | | C5- | C8- | | | | |
| | gel, 3% | XG | LG | GG | GU | diol | diol | Glycerol | (1) | (2) | (3) |
| Comp | 100 | 0 | 0 | 0 | 0 | 0 | 0 | 0 | − | − | − |
| ex. 1 | | | | | | | | | | | |
| Comp | 80 | 0 | 0 | 0 | 0 | 0 | 0.5 | 19.5 | − | + | − |
| ex. 2 | | | | | | | | | | | |
| Comp | 80 | 0 | 0 | 0 | 0 | 20 | 0 | 0 | − | + | − |
| ex. 3 | | | | | | | | | | | |
| Ex. 4 | 80 | 1.0 | 0 | 0 | 0 | 19 | 0 | 0 | + | + | + |
| Ex. 5 | 80 | 0 | 0 | 1.0 | | 19 | 0 | 0 | +/− | + | + |
| Ex.6 | 80 | 0 | 0 | 0 | 1.0 | 19 | 0 | 0 | +/− | + | +/− |
| Ex. 7 | 80 | 1.0 | 0 | 0 | 0 | 0 | 0.5 | 18.5 | + | + | + |
| Ex. 8 | 80 | 0 | 1.0 | 0 | 0 | 0 | 0.5 | 18.5 | + | + | + |
| Ex. 9 | 80 | 0 | 0 | 1.0 | 0 | 0 | 0.5 | 18.5 | + | + | + |
| Ex. 10 | 80 | 0 | 0 | 0 | 1.0 | 0 | 0.5 | 18.5 | + | + | + |
| Ex. 11 | 80 | 0.5 | 0 | 0 | 0 | 19.5 | 0 | 0 | + | + | + |
| Ex. 12 | 80 | 0.5 | 0 | 0 | 0 | 0 | 0.5 | 19 | + | + | + |
| Ex. 13 | 80 | 0 | 0 | 0 | 0.5 | 0 | 0.5 | 19 | + | + | + |
| Ex. 14 | 89 | 0.25 | 0 | 0 | 0 | 0 | 0.5 | 19.25 | + | + | − |
| Ex. 15 | 80 | 0.4 | 0 | 0 | 0 | 0 | 0.5 | 19.1 | + | + | + |
| Ex. 16 | 80 | 0.4 | 0 | 0 | 0 | 19.6 | 0 | 0 | + | + | + |
| Ex. 17 | 89 | 0.5 | 0 | 0 | 0 | 0 | 0.5 | 10 | + | + | + |
| Ex. 18 | 94 | 0.5 | 0 | 0 | 0 | 0 | 0.5 | 5 | + | + | + |
| Ex. 19 | 89.5 | 0.5 | 0 | 0 | 0 | 10 | 0 | 0 | + | + | + |
| Ex. 20 | 94.5 | 0.5 | 0 | 0 | 0 | 5 | 0 | 0 | + | + | + |
| Ex. 21 | 94.6 | 0.4 | 0 | 0 | 0 | 5 | 0 | 0 | + | + | + |
| Ex. 22 | 95.6 | 0.5 | 0 | 0 | 0 | 0 | 0.5 | 2.5 | + | + | + |
| Ex. 23 | 96.6 | 0.4 | 0 | 0 | 0 | 0 | 0.5 | 2.5 | + | + | + |
| Ex. 24 | 94.1 | 0.4 | 0 | 0 | 0 | 0 | 0.5 | 5 | + | + | + |
| Ex. 25 | 96.7 | 0.3 | 0 | 0 | 0 | 0 | 0.5 | 2.5 | + | + | + |

Abbreviations:
MFC gel: aqueous suspension containing 3% microfibrous cellulose
XG: Xanthan Gum E415, Jungbunzlauer, normal granulometry
LG: Locust Bean Gum, Viscogum BE ®/Cargill
GG: Gellan Gum, TayaGel ®/Jungbunzlauer
GU: Guar Gum, Jaguar S ®/Azelis
C5-diol: 1,2-pentanediol, A-Leen 5 ®/Minasolve
C8-diol: 1,2-octanediol, A-Leen 8 ®/Minasolve

It was found that the compositions according to the invention (examples 4-25) generally passed the various stress tests, while the compositions lacking at least one of the components according to the invention (comparative examples 1-3) did not pass all the stress tests.

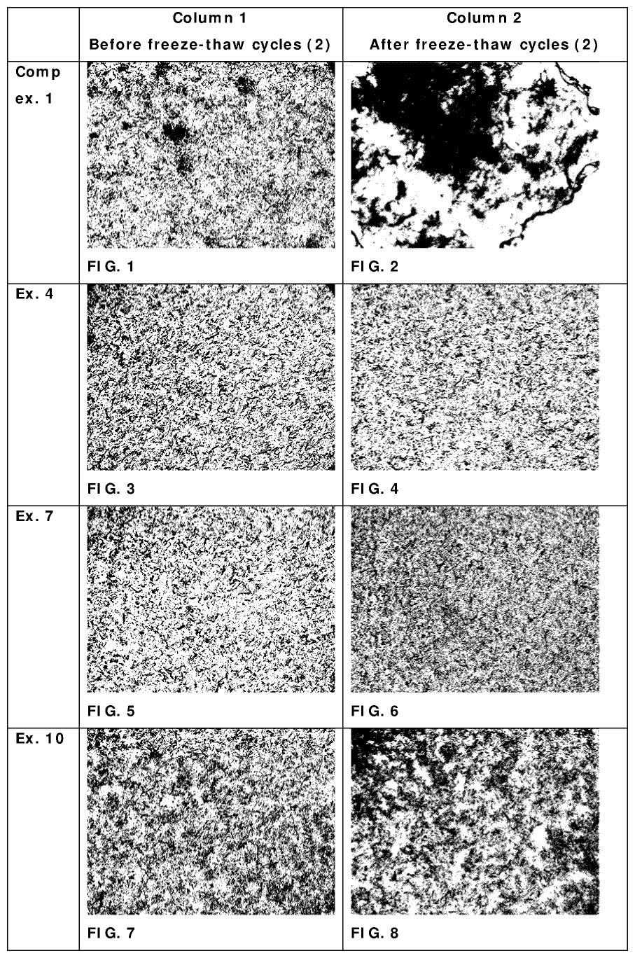

Microscopic pictures of several of the suspensions were taken before and after freeze-thaw cycles of storage at −20° C. for 12 hours, followed by storage at 20° C. for 12 hours. The results are shown in FIGS. 1-8. Column 1, showing FIGS. 1, 3, 5 and 7 were images made before freeze-thaw cycles. Column 2, showing FIGS. 2, 4 6 and 8 were images made after 3 freeze-thaw cycles. The freeze-thaw cycles were carried out according to (2): Three consecutive freeze-thaw cycles of storage at −20° C. for 12 hours, followed by storage at 20° C. for 12 hours.

The microscopic analysis revealed that the three-dimensional network structure of the aqueous MF(H)C gels (example 4, 7, 10) of the invention was preserved, whereas the structure of the comparative example 1 without the stabilizing additives collapsed.

### EXAMPLES 26-29 AND COMPARATIVE EXAMPLES 30-32

Shampoos (examples 26-29 and comparative examples 30-32) according to table 2 were prepared according to the following process:
Disperse MFC Gel (freshly prepared, according to a composition according to table 1) in water until a uniform gel is obtained (<10 min), using a turbine stirrer at 1000 rpm.
Add Xanthan Gum, continue mixing at 1000 rpm until it is fully hydrated
Add Phase B ingredients one at a time, mix between additions with a turbine stirrer at 400 rpm
Add Phase C under mixing with a turbine stirrer at 400 rpm
Adjust pH value to 5-5.5 while mixing with a turbine stirrer at 400 rpm

TABLE 2

| | | | | | | | Comp | Comp | Comp |
|---|---|---|---|---|---|---|---|---|---|
| | Raw | INCI | Ex. | Ex. | Ex. | Ex | ex. | ex. | ex. |
| | Materials | name | 26 | 27 | 28 | 29 | 30 | 31 | 32 |
| A | De- | Water | ad | ad | ad | ad | ad | ad | ad |
| | mineralised | | 100 | 100 | 100 | 100 | 100 | 100 | 100 |
| | water | | | | | | | | |
| | MFC Gel | Water, | 10.0 | 3.3 | 0 | 0 | 0 | 0 | 0 |
| | (Ex. 21) | Pentylene | | | | | | | |
| | | Glycol, | | | | | | | |
| | | Cellulose, | | | | | | | |
| | | Xanthan | | | | | | | |
| | | Gum | | | | | | | |
| | MFC Gel | Water, | 0 | 0 | 10.0 | 3.3 | 0 | 0 | 0 |
| | (Ex. 24) | Glycerol, | | | | | | | |
| | | Cellulose, | | | | | | | |
| | | Caprylyl | | | | | | | |
| | | Glycol, | | | | | | | |
| | | Xanthan | | | | | | | |
| | | Gum | | | | | | | |
| | MFC Gel | Water, | 0 | 0 | 0 | 0 | 0 | 10.0 | 0 |
| | (Comp ex. 1) | Cellulose | | | | | | | |
| | MFC Gel | Water, | 0 | 0 | 0 | 0 | 0 | 0 | 10.0 |
| | (Comp ex. 3) | Pentylene | | | | | | | |
| | | Glycol, | | | | | | | |
| | | Cellulose | | | | | | | |
| | Xanthan | Xanthan | 0.6 | 0.6 | 0.6 | 0.6 | 0.6 | 0.6 | 0.6 |
| | Gum | Gum | | | | | | | |
| B | Plantacare | Coco- | 15.0 | 15.0 | 15.0 | 15.0 | 15.0 | 15.0 | 15.0 |
| | 818 UP ® | Glucoside | | | | | | | |
| | Plantapon | Sodium | 5.0 | 5.0 | 5.0 | 5.0 | 5.0 | 5.0 | 5.0 |
| | ACGHC ® | Cocoyl | | | | | | | |
| | | Glutamate | | | | | | | |
| | Tego Betain | Cocamido- | 5.0 | 5.0 | 5.0 | 5.0 | 5.0 | 5.0 | 5.0 |
| | F50 ® | propyl | | | | | | | |
| | | Betaine | | | | | | | |
| C | E-Leen 8 ® | Caprylyl | 2.0 | 2.0 | 2.0 | 2.0 | 2.0 | 2.0 | 2.0 |
| | | Glycol, | | | | | | | |
| | | Glycerin, | | | | | | | |
| | | Water | | | | | | | |
| D | Citric acid | Citric acid | pH | pH | pH | pH | pH | pH | pH |
| | | | 5.5 | 5.5 | 5.5 | 5.5 | 5.5 | 5.5 | 5.5 |

The shampoos (examples 26-29 and comparative examples 30-32) according to the invention were all obtained as uniform opaque gels without high-shear mixing.

INCI stands for “International Nomenclature of Cosmetic Ingredients”. INCI was developed by the European and American cosmetics industry. INCI names are internationally standardized names for substances processed in cosmetics. On the label, all cosmetics indicate the ingredients by their INCI names.

The shampoos according to examples 28 and 29 were subjected to tests with consumers, which demonstrated a hair-conditioning effect resulting, for example, in easier combing and increased hair volume.

### EXAMPLES 33-34 AND COMPARATIVE EXAMPLES 35-36

Shampoos (examples 33-34 and comparative examples 35-36) according to table 3 were prepared according to the process as described for examples 26-29 and comparative examples 30-32, but instead of freshly prepared MFC Gel, an MFC gel subjected to three consecutive freeze-thaw cycles (2) is used.

TABLE 3

| | | | | | Comp | Comp |
|---|---|---|---|---|---|---|
| | Raw Materials | INCI name | Ex. 33 | Ex. 34 | ex. 35 | ex. 36 |
| A | Demineralised | Water | ad 100 | ad 100 | ad 100 | ad 100 |
| | water | | | | | |
| | MFC Gel | Water, | 10.0 | 0 | 0 | 0 |
| | (Ex. 21) (2) | Pentylene | | | | |
| | | Glycol, | | | | |
| | | Cellulose, | | | | |
| | | Xanthan Gum | | | | |
| | MFC Gel | Water, Glycerol, | 0 | 10.0 | 0 | 0 |
| | (Ex. 24) (2) | Cellulose, | | | | |
| | | Caprylyl Glycol, | | | | |
| | | Xanthan Gum | | | | |
| | MFC Gel | Water, Cellulose | 0 | 0 | 10.0 | 0 |
| | (Comp ex. 1) | | | | | |
| | (2) | | | | | |
| | MFC Gel | Water, | 0 | 0 | 0 | 10.0 |
| | (Comp ex. 3) | Pentylene | | | | |
| | (2) | Glycol, | | | | |
| | | Cellulose | | | | |
| | Xanthan Gum | Xanthan Gum | 0.6 | 0.6 | 0.6 | 0.6 |
| B | Plantacare 818 | Coco-Glucoside | 15.0 | 15.0 | 15.0 | 15.0 |
| | UP ® | | | | | |
| | Plantapon | Sodium Cocoyl | 5.0 | 5.0 | 5.0 | 5.0 |
| | ACGHC ® | Glutamate | | | | |
| | Tego Betain | Cocamidopropyl | 5.0 | 5.0 | 5.0 | 5.0 |
| | F50 ® | Betaine | | | | |
| C | E-Leen 8 ® | Caprylyl Glycol, | 2.0 | 2.0 | 2.0 | 2.0 |
| | | Glycerin, Water | | | | |
| D | Citric acid | Citric acid | pH 5.5 | pH 5.5 | pH 5.5 | pH 5.5 |

The shampoos from examples 33 and 34 and comparative example 36 were obtained as uniform opaque gels without high shear mixing.

Comparative example 35 contained some lumps when prepared without high shear mixing.

### EXAMPLES 37-40 AND COMPARATIVE EXAMPLE 41

The shampoos prepared in examples 26-29 and comparative example 30 (5 g) were dissolved in demineralized water (45 g) by slow mixing with a spatula without producing foam. The solutions (examples 37-40 and comparative example 41) were gently poured into a separating funnel. The tap of the funnel was opened, and the solution was drained from a height of 40 cm into a measuring cylinder. The volume of foam formed in the cylinder was measured after 0 min and 10 min and is shown in table 4.

TABLE 4

| Foam volume | Ex. 37 | Ex. 38 | Ex. 39 | Ex. 40 | Comp ex. 41 |
|---|---|---|---|---|---|
| Dissolved | Ex. 26 | Ex. 27 | Ex. 28 | Ex. 29 | Comp ex. 30 |
| shampoo | | | | | |
| 0 min | 27 mL | 28 mL | 26 mL | 34 mL | 8 mL |
| 10 min | 21 mL | 24 mL | 22 mL | 30 mL | 5 mL |

Relative to the comparative examples, the foam obtained with the shampoos containing stable suspensions according to the invention was more voluminous, more stable and had smaller bubbles, resulting in a creamier consistency of the foam.

### EXAMPLE 42-43 AND COMPARATIVE EXAMPLE 44

Oil in water emulsions (examples 42-43 and comparative examples 44) according to table 5 were prepared according to the following process:
Disperse MFC Gel in water.
Add Xanthan Gum and mix for 25 min at 700-800 rpm then heat mixture up to 75-80° C.
Mix and heat up all ingredients forming phase B to 80° C.
Add B to A while stirring when they reached the same temperature. Homogenize for 3 min by Ultra Turrax (9500-13500 rpm), then continue mixing with normal stirrer for 30 min at 1000 rpm. Keep mixing while the cream is cooling down.
Add Tocopherol at <40° C. Mix until homogeneous. Add E-Leen Green B.
Adjust the pH

TABLE 5

| | | | Ex. | Ex. | Comp |
|---|---|---|---|---|---|
| | | | 42 | 43 | ex. 44 |
| Phase | Ingredient | INCI name | % | % | % |
| A | Water | Water | Ad 100 | Ad 100 | Ad 100 |
| | MFC Gel | Water, Pentylene Glycol, | 18.5 | 0 | 0 |
| | (Ex. 21) | Cellulose, Xanthan Gum | | | |
| | MFC Gel | Water, Glycerol, Cellulose, | 0 | 18.5 | 0 |
| | (Ex. 24) | Caprylyl Glycol, Xanthan Gum | | | |
| | Xanthan Gum | Xanthan Gum | 0.6 | 0.6 | 0.6 |
| B | Emulgade PL | Cetearyl Glucoside (and) | 5 | 5 | 5 |
| | 68/50 | Cetearyl Alcohol | | | |
| | Shea Butter | Butyrospermum Parkii (Shea) | 3 | 3 | 3 |
| | | Butter | | | |
| | Jojoba oil | Simmondsia Chinensis | 3 | 3 | 3 |
| | | (Jojoba) Seed Oil | | | |
| | Apricot oil | Prunus Armeniaca (Apricot) | 3 | 3 | 3 |
| | | Kernel Oil | | | |
| C | Tocopherol | Tocopherol | 0.1 | 0.1 | 0.1 |
| D | E-Leen Green B | Pentylene Glycol, Water, | 2 | 2 | 2 |
| | | Sodium Benzoate, | | | |
| | | Benzoic Acid | | | |
| E | Citric acid | Citric acid | pH 5.5 | pH 5.5 | pH 5.5 |

The following results were obtained by a panel of 5 experts:
The emulsions of examples 42 and 43 appeared white while the comparative emulsion 44 was light beige.
The emulsions of examples 42 and 43 appeared to provide a less oily skin feel than the comparative emulsion 44.
The emulsions of examples 42 and 43 appeared to provide a richer texture than the comparative emulsion 44.

### EXAMPLES 45-46 AND COMPARATIVE EXAMPLE 47

BB creams with pigments (examples 45-46 and comparative examples 47) according to table 6 were prepared.

TABLE 6

| | | | Ex. | Ex. | Comp |
|---|---|---|---|---|---|
| | | | 45 | 46 | ex. 47 |
| | Ingredient | INCI name | % | % | % |
| A | Water | Water | ad 100 | ad 100 | ad 100 |
| | Glycerin | Glycerin | 5.0 | 5.0 | 5.0 |
| | Xanthan Gum | Xanthan Gum | 0.4 | 0.4 | 0.4 |
| | Gummi | Gum Arabic | 1.8 | 1.8 | 1.8 |
| | Arabicum | | | | |
| | MFC Gel #21 | Water, Pentylene Glycol, | 18.5 | 0 | 0 |
| | | Cellulose, Xanthan Gum | | | |
| | MFC Gel #24 | Water, Glycerol, Cellulose, | 0 | 18.5 | 0 |
| | | Caprylyl Glycol, Xanthan Gum | | | |
| B | Myritol 318 | Caprylic/Capric Triglyceride | 17.7 | 17.7 | 17.7 |
| | Emulgade PL | Cetearyl Glucoside (and) | 6.8 | 6.8 | 6.8 |
| | 68/50 | Cetearyl Alcohol | | | |
| | Pigments | Titanium dioxide (CI 77891), | 8.0 | 8.0 | 8.0 |
| | | Iron Oxide Yellow (CI 77492), | | | |
| | | Iron Oxide Brown (CI 77499) | | | |
| | A-Leen 5 | Pentylene Glycol | 2.0 | 2.0 | 2.0 |
| C | E-Leen Green B | Pentylene Glycol, Water, | 3.0 | 3.0 | 3.0 |
| | | Sodium Benzoate, Benzoic Acid | | | |
| D | Citric Acid | Citric Acid | pH 5.5 | pH 5.5 | pH 5.5 |

The creams of examples 45 and 46 caused a more intense color on the skin than the comparative emulsion 47.

### EXAMPLE 48-49

The suspensions according to examples 21 and 24 were subjected to microbial challenge tests according to the ISO 11930 standard, examples 48 and 49 respectively. The observed log reductions of microbial counts over time are summarized in Table 7. In summary, the products tested showed good resistance to microbial attack and passed the challenge test. It was therefore shown that the suspensions according to the invention are generally self-preserving without the need to add further preservatives.

TABLE 7

| | Ex. 48 | | | Ex. 49 | | |
|---|---|---|---|---|---|---|
| | Susp. according to Ex. 21 | | | Susp. according to Ex. 24 | | |
| Germ | Day 7 | Day 14 | Day 28 | Day 7 | Day 14 | Day 28 |
| Pseudomonas | >4.6 | >4.6 | >4.6 | 1.0 | >4.6 | >4.6 |
| aeruginosa | | | | | | |
| Staphylococcus | 3.3 | >4.6 | >4.6 | >4.6 | >4.6 | >4.6 |
| aureus | | | | | | |
| Escherichia | >4.7 | >4.7 | >4.7 | >4.7 | >4.7 | >4.7 |
| coli | | | | | | |
| Candida | 0.9 | >3.4 | >3.4 | >3.4 | >3.4 | >3.4 |
| albicans | | | | | | |
| Aspergillus | — | 0.1 | 0.2 | — | 3.2 | >3.5 |
| brasiliensis | | | | | | |

### EXAMPLES 50-51 AND COMPARATIVE EXAMPLES 52-55

The stable suspensions of examples 50 and 51 were prepared by the following process: The MFC gel is poured into a beaker. The natural gum (dry powder) and the liquid polyols are carefully premixed in a separate beaker until a uniform dispersion is obtained. The gum/polyol premix is added to the MFC gel. The resulting mixture is homogenized for 3 minutes at 10,000 rpm using a Silverson L5M-A mixer equipped with a general-purpose disintegrating workhead.

The suspensions of comparative examples 52 and 53 were prepared by the following process: The MFC gel is poured into a beaker. The natural gum is added as a powder, followed by the liquid polyols. The resulting mixture is homogenized for 3 minutes at 10,000 rpm using a Silverson L5M-A mixer equipped with a general-purpose disintegrating workhead.

The suspensions of comparative examples 54 and 55 were prepared by the following process: The MFC gel is poured into a beaker. The liquid polyols are added, followed by the natural gum as a dry powder. The resulting mixture is homogenized for 3 minutes at 10,000 rpm using a Silverson L5M-A mixer equipped with a general-purpose disintegrating workhead.

Table 8 shows that the suspensions obtained according to the production method of the invention were all obtained as uniform opaque gels, while inhomogeneous gels with lumps were obtained when the polyol and the thickening agent are not pre-mixed. In this case much longer mixing times are necessary to obtain a stable suspension. The inventors have unexpectedly found that the addition of the thickener without pre-mixing with a polyol leads to lump formation or to extended mixing times. The invention therefore provides a solution to efficiently mix a natural thickener into a thick paste of MF(H)C, thereby avoiding lump formation and achieving short production times.

TABLE 8

| | | Xanthan | | |
|---|---|---|---|---|
| Ex. # | MFC Gel | Gum | Polyols | Aspect of the mixture |
| Ex. 50 | 945 g | 5 g | 50 g | Uniform opaque gel |
| | | | Pentylene | |
| | | | Glycol | |
| Ex. 51 | 945 g | 5 g | 45 g | Uniform opaque gel |
| | | | Glycerol | |
| | | | 5 g | |
| | | | Caprylyl | |
| | | | Glycol | |
| Comp | 945 g | 5 g | 50 g | Inhomogeneous gel with |
| ex. 52 | | | Pentylene | lumps |
| | | | Glycol | |
| Comp | 945 g | 5 g | 45 g | Inhomogeneous gel with |
| ex. 53 | | | Glycerol | lumps |
| | | | 5 g | |
| | | | Caprylyl | |
| | | | Glycol | |
| Comp | 945 g | 5 g | 50 g | Inhomogeneous gel with |
| ex. 54 | | | Pentylene | lumps |
| | | | Glycol | |
| Comp | 945 g | 5 g | 45 g | Inhomogeneous gel with |
| ex. 55 | | | Glycerol | lumps |
| | | | 5 g | |
| | | | Caprylyl | |
| | | | Glycol | |

The present invention is in no way limited to the embodiments described in the examples. On the contrary, methods according to the present invention may be realized in many different ways without departing from the scope of the invention.

1. A stable suspension comprising:
microfibrous cellulose, microfibrous hemicellulose or mixtures thereof (MF(H)C), in an amount of 1 to 10 percent by weight of the stable suspension;
at least one natural thickening agent, in an amount up to 2 percent by weight of the stable suspension;
at least one polyol, in an amount up to 20 percent by weight of the stable suspension; and
water, in an amount of 50 to 98 percent by weight of the stable suspension.

2. The stable suspension according to claim 1, wherein the stable suspension comprises the microfibrous cellulose, the microfibrous hemicellulose or the mixtures thereof (MF(H)C), in an amount of 1 to 5 percent by weight of the stable suspension.

3. The stable suspension according to claim 1, wherein the stable suspension comprises at least one natural thickening agent, in an amount up to 1 percent by weight of the stable suspension.

4. The stable suspension according to claim 1, wherein said MF(H)C is microfibrillated from cellulose and/or hemicellulose fibers by a process comprising only a physical application of high-shear forces and does not involve any chemical, biotechnological, or enzymatic conversions.

5. The stable suspension according to claim 1, wherein fibers of said microfibrous cellulose and/or said microfibrous hemicellulose are natural cellulose fibers and/or hemicellulose fibers.

6. The stable suspension according to claim 1, wherein the stable suspension has the form of a homogeneous suspension.

7. The stable suspension according to claim 1, wherein said at least one natural thickening agents are poly-saccharide-based thickeners.

8. The stable suspension according to claim 1, wherein the at least one polyol is selected from diols or triols containing a branched or non-branched chain of 3 to 12 carbon atoms or a mixture thereof.

9. The stable suspension according to claim 1, wherein the at least one polyol is selected from glycerol, 1,2-propanediol, 1,3-propanediol, butylene glycol, 1,2-pentanediol, isopentyldiol, 1,2-hexanediol, hexylene glycol, 1,2-heptanediol, 1,2-octanediol, 1,2-nonanediol, 1,2-decanediol, 1,2-undecanediol, methylheptylglycerin, ethylhexylglycerin, 1,2-dodecanediol or a mixture thereof.

10. The stable suspension according to claim 1, wherein the stable suspension comprises the at least one polyol and the at least one natural thickening agent in a ratio by weight of the between 80/1 and 6/1.

11. A method for producing a stable suspension according to claim 1, comprising:
providing an aqueous suspension comprising MIF(H)C,
dispersing at least one natural thickening agent in at least one polyol to form a dispersion, and
adding the dispersion to the aqueous suspension comprising MF(H)C under mixing.

12. A use of a stable suspension according to claim 1, in a cosmetic, pharmaceutical, dermatological or hygienic preparation.

13. A cosmetic product, a pharmaceutical product, a dermatological product, or a hygienic product comprising a stable suspension according to claim 1.

14. A cosmetic product, a pharmaceutical product, a dermatological product, or a hygienic product comprising a stable suspension according to claim 1, wherein said products comprise the stable suspension in an amount of between 1 and 30 percent by weight of said products.

15. A method for producing the cosmetic product, the pharmaceutical product, the dermatological product, or the hygienic product according to claim 13, wherein the method does not comprise the step of high shear mixing.
